(19) United States
(12) Patent Application Publication
(10) Pub. No.: US 2023/0257501 A1
(43) Pub. Date: Aug. 17, 2023

(54) NOVEL COPOLYMERS AND THEIR USE IN PHARMACEUTICAL DOSAGE FORMS

(71) Applicant: BASF SE, Ludwigshafen (DE)

(72) Inventors: Theo Smit, Ludwigshafen (DE); Ferdinand Paul Brandl, Ludwigshafen (DE); Felicitas Guth, Ludwigshafen (DE); Karl Kolter, Limburgerhof (DE); Frank Schmidt, Hochdorf-Assenheim (DE)

(21) Appl. No.: 18/014,508

(22) PCT Filed: Jul. 2, 2021
(86) PCT No.: PCT/EP2021/068283

§ 371 Date: Jan. 5, 2023

(30) Foreign Application Priority Data
Jul. 9, 2020 (EP) 20184950.2

(51) Int. Cl. C08F 226/10; C08F 2/06; A61K 9/16

(52) U.S. Cl. CPC C08F 226/10; C08F 2/06; A61K 9/1635

(57) ABSTRACT

Copolymer, wherein structural units are derived from: i) an acrylic carboxylic acid monomer (4 to 18% by weight), selected from the group consisting of acrylic acid and methacrylic acid, ii) a 5 hydrophobic methacrylate (more than 8% by weight), selected from a group consisting of isopropyl methacrylate, tert-butyl methacrylate and cyclohexyl methacrylate, iii) a N-vinyl lactam, selected from a group consisting of N-vinyl pyrrolidone and N-vinylcaprolactam and optionally iv) 2-hydroxyethyl methacrylate, with the proviso that the total amount of structural units derived from the monomer groups adds up to 100% by weight, and the calculated solubility parameter SP of 10 the copolymer is between 22.0 and 25.0 MPa^{1/2}, and the use of the copolymers as crystallization inhibitors in pharmaceutical dosage forms for inhibiting the recrystallization of an active ingredient in an aqueous environment of a human or animal body.

The present invention relates to novel copolymers based on a hydrophobic methacrylate monomer, an N-vinyl lactam monomer, an olefinic carboxylic acid monomer and optionally a hydroxyethyl methacrylate monomer, their use as pharmaceutical excipients for improving gastrointestinal absorption, the respective pharmaceutical dosage forms and methods for making the copolymers

The intestinal absorption of poorly water-soluble drugs (BCS class II and IV) is limited by the maximum achievable concentration in the gastrointestinal lumen. Therefore, various approaches in formulation development aim at increasing the dissolution rate and improving drug solubility in the gastrointestinal tract. Administering the drug as a solution is a common approach to enhance the intestinal absorption of poorly water-soluble drugs. To this end, hydrophobic drugs are formulated using a mixture of co-solvents, surfactants, complexing agents (e.g., cyclodextrins) and/or oils. After oral administration, these formulations increase the total concentration of the drug that is present in solution; however, this approach does not necessarily result in an improved bioavailability. Depending on the lipophilicity of the drug, a large fraction of drug molecules is solubilized in a mixture of colloidal species (e.g., emulsified oil, micelles etc.). This fraction is unavailable for absorption, since only the free molecular species of the drug can permeate across the intestinal barrier. Furthermore, dilution and dispersion of the formulation in the gastrointestinal tract decreases the solubilization capacity. As a result, a metastable supersaturated state is generated that eventually leads to drug precipitation.

Besides the administration in solution, a number of formulation strategies exist that enable the delivery of poorly water-soluble drugs in a solid form. These approaches aim at generating high-energy or rapidly dissolving forms of the drugs (e.g., by milling, co-grinding, solvent evaporation, melting or crystal engineering) that induce supersaturation in the gastrointestinal tract. For example, in combination with suitable polymers (e.g., polyvinylpyrrolidone, vinylpyrrolidone-vinyl acetate copolymer, polyethylene glycol, polymethacrylates, cellulose derivatives etc.) and/or surfactants, poorly water-soluble drugs can be manufactured into solid dispersions (e.g., by spray drying or hot melt extrusion). These contain amorphous drug particles embedded in a polymer matrix that stabilizes the amorphous state by vitrification, specific drug-polymer interactions and/or reduced mobility. The release of the embedded drug molecules often depends on the dissolution rate of the polymer matrix. After dissolution of the dosage form in the gastrointestinal tract, the concentration of the drug in solution will be above the saturation solubility. This supersaturated state is thermodynamically unstable and the system tends to return to the equilibrium state by drug precipitation.

To benefit from the increased concentration, it is necessary to stabilize the supersaturated state in the gastrointestinal lumen for a time period sufficient for absorption to take place. For example, cellulose derivatives (e.g., hydroxypropyl methylcellulose (HPMC) or hydroxypropyl methylcellulose acetate succinate (HPMCAS)) and vinyl polymers (e.g., polyvinyl alcohol, polyvinylpyrrolidone or vinylpyrrolidone-vinyl acetate copolymer) have been described to inhibit drug precipitation by interfering with nucleation and/or crystal growth. It is important to note that this type of stabilization in solution is different from the stabilization of the amorphous state in the dosage form prior to application.

WO2014/159748 mentions the use of acrylate based crystallization-inhibiting agents, preferably a copolymer of butyl methacrylate, 2-dimethylaminethyl methacrylate and methyl methacrylate in a weight ratio 1:2:1

WO 2005/058383 describes adhesive implants for parietal repair comprising water-soluble biocompatible polymers having adhesive properties which are copolymers based on alkyl acrylates such as octyl acrylates as well as acrylic acid and hydroxyalkyl (meth)acrylates.

WO 2014/182713 relates to statistical copolymers made from at least three different acrylate monomers such as alkyl(meth)acrylate, carbalkoxyalkyl (meth)acrylates, hydroxyalkyl (meth)acrylates and alkyl acetyl acrylates and their use for inhibiting drug crystallization and supersaturation maintenance. WO 2014/182710 refers to similar copolymers further substituted with sugar moieties. These copolymers show several disadvantages. First of all, not all of the monomer groups are readily available and need to be specifically synthesized. Another problem is that the copolymers have relatively low glass transition temperatures which makes spray drying difficult. Lower glass transition temperatures are also disadvantageous with regard to storage stability because the dosage forms tend to the so-called “cold flow”. Also, sugar substituted copolymers show instabilities when processed by melt-extrusion.

The acrylic copolymers described in WO 2019/121051 fulfil most of the requirements for crystallization inhibition but suffer from the drawback, that the carboxylic acid groups need to be partly neutralized to obtain polymers that are sufficiently soluble in intestinal fluid. The resulting alkali metal carboxylate groups are hygroscopic. Softening of the polymer matrix by water uptake increases the risk of API (active pharmaceutical ingredient) crystallization in polymer/API amorphous solid dispersions.

According to the prior art, cellulose derivatives such as HPMCAS are often considered the excipient of choice to inhibit drug precipitation [J. Brouwers, M. E. Brewster, P. Augustijns, Supersaturating drug delivery systems: The answer to solubility-limited oral bioavailability? Journal of Pharmaceutical Sciences, 98 (2008) 2549-2572; D. B. Warren, H. Benameur, C. J. H. Porter, C. W. Pouton, Using polymeric precipitation inhibitors to improve the absorption of poorly water-soluble drugs: A mechanistic basis for utility, Journal of Drug Targeting, 18 (2010) 704-731; S. Baghel, H. Cathcart, N.J. O'Reilly, Polymeric amorphous solid dispersions: A review of amorphization, crystallization, stabilization, solid-state characterization, and aqueous solubilization of biopharmaceutical classification system class II drugs, Journal of Pharmaceutical Sciences, 105 (2016) 2527-2544]. However, the effectiveness of HPMCAS and other known polymers is often not ideal, since these polymers have originally been optimized for other applications (e.g., coatings or thickening agents). So far, there are no excipients available that fulfill all requirements regarding variability, long-term stability, processability and performance.

The problem to be solved by the present invention was to develop excipients for pharmaceutical formulations that allow for a safe and efficient stabilization against recrystallization and precipitation from the supersaturated state after in vivo release of sparingly water-soluble active ingredients in the aqueous environment of the human or animal body in order to assure satisfactory bioavailability.

The problem was solved by finding copolymers consisting of: i) not less than 8% by weight on the total amount of all incorporated monomers of a hydrophobic methacrylate, ii) an N-vinyl lactam monomer, iii) 4 to 18% by weight on the total amount of all incorporated monomers of an acrylic carboxylic acid monomer and optionally iv) 2-hydroxyethyl methacrylate, with the proviso that the amount of incorporated monomers i) to iv) adds up to 100% by weight and the calculated solubility parameter SP of the copolymer is between 22.0 and 25.0 MPa^{1/2}. These polymers do not require a post-polymerization neutralization of the incorporated carboxylic acid groups to become sufficiently soluble in fasted state simulated intestinal fluid at pH 6.8.

According to a preferred embodiment, the invention relates to copolymers consisting of: i) 8 to 55% by weight on the total amount of all incorporated monomers of a hydrophobic methacrylate, ii) 10 to 88% by weight on the total amount of all incorporated monomers of an N-vinyl lactam monomer, iii) 4 to 18% by weight on the total amount of all incorporated monomers of an acrylic carboxylic acid monomer and iv) 0 to 40% by weight on the total amount of all incorporated monomers of 2-hydroxyethyl methacrylate, with the proviso that the amount of incorporated monomers i) to iv) adds up to 100% by weight and the calculated solubility parameter SP of the copolymer is between 22.0 and 25.0 MPa^{1/2}.

In accordance with the present invention the hydrophobic methacrylate monomer i) can be selected of tert-butyl methacrylate, isopropyl methacrylate, cyclohexyl methacrylate and mixtures thereof, ii). the N-vinyl lactam monomer can be selected of N-vinylpyrrolidone and N-vinylcaprolactam and mixtures thereof and iii). the acrylic carboxylic acid monomer can be selected of acrylic acid and methacrylic acid and mixtures thereof.

Another aspect of the invention is the use of the copolymers for inhibiting in vivo recrystallization of an active ingredient after release from a dosage form into the aqueous environment of the human or animal body and the respective dosage forms comprising the copolymer and an active ingredient, wherein the active ingredient has a solubility in water under standard conditions (temperature of 23° C. and a pressure of 0.101325 MPa) of less than 0.1% by weight. Preferably, the solubility of the active ingredient in water under standard conditions is less than 0.05% by weight, the active ingredient being present in such dosage form in an amorphous state or molecularly dispersed. Amorphous means that less than 5% by weight are crystalline. The crystalline proportion can be measured by X-Ray diffraction methods.

In accordance with the present invention solubility whether in water, phosphate buffer or other suitable biologically relevant systems is always the solubility at standard conditions, i.e., a temperature of 23° C. and a pressure of 0.101325 MPa.

According to the invention, active ingredients sparingly soluble in water are those having a solubility of less than 0.1% by weight in water at standard conditions.

As already mentioned, suitable copolymers for water-soluble formulations of active ingredients sparingly soluble in water, are those copolymers having a solubility in fasted state simulated intestinal fluid at a pH of 6.8 at 37° C. as defined above which are obtained by free-radically initiated polymerization of a mixture of different monomers to give a copolymer consisting of: i) not less than 8% by weight on the total amount of all incorporated monomers of a hydrophobic methacrylate, ii) an N-vinyl lactam monomer, iii) 4 to 18% by weight on the total amount of all incorporated monomers of an acrylic carboxylic acid monomer and optionally iv) hydroxyethyl methacrylate, and with an calculated solubility parameter SP between 22.0 and 25.0 MPa^{1/2}. The sparingly water-soluble active ingredient has a solubility of less than 0.1% by weight in water, artificial intestinal juice or gastric juice.

In all embodiments of the invention the amounts for the monomer derived moieties given in percent by weight are meant to include a deviation of ±1% by weight.

The polymers can be prepared in a conventional manner per se by free-radical polymerization.

The polymerization is preferably carried out as a solution polymerization in organic solvents, preferably in alcohols such as methanol or ethanol, particularly in isopropanol. Such methods are known per se to those skilled in the art. Suitable initiators are, for example, organic peroxides such as diisobutyryl peroxide, 1,1,3,3-tetramethylbutyl peroxyneodecanoate, tert-amyl peroxyneodecanoate, tert-butyl peroxyneodecanoate, tert-butyl peroxyneoheptanoate, tert-butyl peroxypivalate, tert-butyl peroxy-2-ethylhexanoate and di-tert-butyl peroxide. Preferred are tert-butyl peroxypivalate, tert-butyl peroxy-2-ethylhexanoate and tert-butyl peroxyneodecanoate. Alternatively, alcohol-soluble azo initiators, such as dimethyl-2,2′-azobis(2-methylpropionate) or 2,2′-azobis(2-methylbutyronitrile), can be used to initiate the polymerization.

The polymerization may be conducted at temperatures of from 20 to 150° C., preferably 50 to 130° C. The polymerization can be carried out both under atmospheric pressure or in a closed reactor under elevated pressure. In this case it is possible to polymerize either under the pressure set up during the reaction, or the pressure can be adjusted by injecting a gas or evacuating.

It is also possible to carry out the polymerization in the presence of chain transfer agents, for example 1-dodecanethiol.

The polymerization can be carried out continuously, semi-batch or as a batch process, the polymers preferably being obtained via a feed process.

The conversion of the polymer solutions into the solid form may be carried out by conventional drying processes such as spray-drying, freeze-drying or roller drying.

According to a preferred embodiment, the organic reaction solution of the polymers is directly processed with active ingredients to give solid dispersions.

The weight average molecular weight of the copolymers measured by gel permeation chromatography lies in the range of 7,000 to 100,000 g/mol, preferably 7,000 to 80,000 g/mol and most preferably 10,000 to 70,000 g/mol.

The glass transition temperatures calculated according to the Fox equation are in the range of >80° C., preferably higher than 100° C. and up to 150° C.:

$$\frac{1}{{T}_{G}} = {\sum }_{i}^{n} {x}_{i} \frac{1}{{T}_{G , i}}$$

xᵢ=mass fraction of the comonomer in the polymer

T_{G,i}=glass transition temperature of the homopolymer of the corresponding comonomer

T_{G}=glass transition temperature of the copolymer

The glass transition temperatures may also be measured by differential scanning calorimetry at a heating rate of 20 K/min. The measurements can be performed according to DIN EN ISO 11357-2

The solubility parameter components (δ_{d}, δₚ and δₕ) are calculated from group contributions according to the method of Hoftyzer and Van Krevelen [D. W. Van Krevelen, K. Te Nijenhuis, Cohesive properties and solubility, in: Properties of Polymers (Fourth Edition), Elsevier, Amsterdam, 2009, pp. 189-227]:

$${δ}_{d} = \frac{Σ {F}_{di}}{V} {δ}_{p} = \frac{\sqrt{Σ {F}_{p i}^{2}}}{V} {δ}_{h} = \sqrt{\frac{Σ {E}_{h i}}{V}}$$

δ_{d}=Partial contribution corresponding to dispersive interactions

δₚ=Partial contribution corresponding to polar interactions

δₕ=Partial contribution corresponding to hydrogen bonding interactions

F_{di}=Group contributions to the dispersion component

Fₚᵢ=Group contributions to the polar component

Eₕᵢ=Group contributions to the hydrogen bonding energy

V=Molar volume of the compound

The molar volume of the compound (V) is calculated from group contributions according to the method of Fedors [R. F. Fedors, A method for estimating both the solubility parameters and molar volumes of liquids, Polymer Engineering & Science, 14 (1974) 147-154]. The overall solubility parameter (6) is calculated from the individual components:

δ=\sqrt{δ_{d}²+δₚ²+δₕ²}

The F_{di}, Fₚᵢ², Eₕᵢ and V values for the individual structural groups that were taken from the given references and that were used for the calculation of the overall solubility parameter are listed in Table 1. An exemplary calculation of solubility parameters for a polymer (tBMA-AA-NVP copolymer, 35:10:55) is shown in Table 2 (monomer abbreviations are given in Table 3). The frequency of the structural groups is calculated as follows. The occurrence of each structural group is determined separately for each monomer and multiplied with the molar fraction of the monomer. The individually calculated values are then added to yield the overall frequency of the structural group as shown in Table 1.

TABLE 1
Structural group values given by Hoftyzer and Van
Krevelen for F_{di}, Fₚᵢ², Eₕᵢ and by Fedors for V.

| | F_{di} | Fₚᵢ² | Eₕᵢ | V |
|---|---|---|---|---|
| Group | (MJ/m³)^{1/2} · mol⁻¹ | (MJ/m³)^{1/2} · mol⁻¹ | (J/mol) | (cm³/mol) |
| —CH₃ | 420 | 0 | 0 | 33.5 |
| —CH₂— | 270 | 0 | 0 | 16.1 |
| >CH— | 80 | 0 | 0 | −1.0 |
| >C< | −70 | 0 | 0 | −19.2 |
| —OH | 210 | 250000 | 20000 | 10.0 |
| —CO— | 290 | 592900 | 2000 | 10.8 |
| —COOH | 530 | 176400 | 10000 | 28.5 |
| —COO— | 390 | 240100 | 7000 | 18.0 |
| >N— | 20 | 640000 | 5000 | −9.0 |
| Ring (≥5 atoms) | 190 | 0 | 0 | 16.0 |

The active ingredients can be selected from the group of pharmaceutical, nutritional or agrochemical actives.

Examples which may be mentioned here include antihypertensives, vitamins, cytostatics, especially taxol, anesthetics, neuroleptics, antidepressants, antibiotics, antimycotics, fungicides, chemotherapeutics, urologics, platelet aggregation inhibitors, sulfonamides, spasmolytics, hormones, immunoglobulins, sera, thyroid therapeutics, psychopharmaceuticals, Parkinson's drugs and other antihyperkinetics, ophthalmics, neuropathy preparations, calcium metabolism regulators, muscle relaxants, narcotics, antilipemics, liver therapeutics, coronary drugs, cardiac drugs, immunotherapeutics, regulatory peptides and their inhibitors, hypnotics, sedatives, gynecological drugs, gout remedies, fibrinolytics, enzyme preparations and transport proteins, enzyme inhibitors, emetics, weight-loss drugs, perfusion promoters, diuretics, diagnostics, corticoids, cholinergics, biliary therapeutics, antiasthmatics, broncholytics, beta-receptor blockers, calcium antagonists, ACE inhibitors, arteriosclerosis remedies, antiphlogistics, anticoagulants, antihypotensives, antihypoglycemics, antihypertensives, antifibrinolytics, antiepileptics, antiemetics, antidotes, antidiabetics, antiarrhythmics, antianemics, antiallergics, anthelmintics, analgesics, analeptics, aldosterone antagonists or antiviral active ingredients or active ingredients for the treatment of HIV infections and AIDS syndrome.

TABLE 2
Calculation of solubility parameters for a copolymer consisting of 35
wt % (28 mol %) tBMA, 10 wt % (16 mol %) AA and 55 wt % (56 mol %) NVP
using the group contribution method of Hoftyzer and Van Krevelen.

| | | F_{di} | Fₚᵢ² | Eₕᵢ | V |
|---|---|---|---|---|---|
| Group | Frequency | (MJ/m³)^{1/2} · mol⁻¹ | (MJ/m³)^{1/2} · mol⁻¹ | (J/mol) | (cm³/mol) |
| —CH₃ | 1.12 | 470 | 0 | 0 | 37.5 |
| —CH₂— | 2.69 | 726 | 0 | 0 | 43.3 |
| >CH— | 0.72 | 58 | 0 | 0 | −0.7 |
| >C< | 0.56 | −39 | 0 | 0 | −10.7 |
| —OH | 0.00 | 0 | 0 | 0 | 0.0 |
| —CO— | 0.56 | 163 | 333501 | 1125 | 6.1 |
| —COOH | 0.16 | 84 | 27824 | 1577 | 4.5 |
| —COO— | 0.28 | 109 | 67174 | 1958 | 5.0 |
| >N— | 0.56 | 11 | 359994 | 2812 | −5.1 |
| Ring (≥5 atoms) | 0.56 | 107 | 0 | 0 | 9.0 |

δ_{d} = 19.00 (MJ/m³)^{1/2}
δₚ = 10.00 (MJ/m³)^{1/2}
δₕ = 9.17 (MJ/m³)^{1/2}
δ = 23.35 (MJ/m³)^{1/2}

Preference is given to using the inventive copolymers for preparing formulations with active ingredients sparingly soluble in water.

The formulations can be either real solutions in which both the active ingredient and the inventive copolymer are dissolved in a suitable solvent or mixture of solvents, or solid dispersions in which the active ingredient is embedded in the solid polymer matrix in amorphous form. Solid dispersions are dispersions of one or more active ingredients in a solid polymer matrix [W. L. Chiou, S. Riegelman, Pharmaceutical applications of solid dispersion systems, Journal of Pharmaceutical Sciences, 60 (1971) 1281-1302]. Solid dispersions can be prepared by heating a physical mixture of the active ingredient and the polymer until it melts, followed by cooling and solidification (melting method). Alternatively, solid dispersions can be prepared by dissolving a physical mixture of the active ingredient and the polymer in a common solvent, followed by evaporation of the solvent (solvent method). Solid dispersions may contain the active ingredient molecularly dispersed in a crystalline matrix. Alternatively, solid dispersions may consist of an amorphous carrier; the active ingredient can be either molecularly dispersed in the carrier or form an amorphous precipitate. In any case, the active ingredient needs to be in an amorphous form. “Amorphous” means that less than 5% by weight of the active ingredient are crystalline.

According to one embodiment of the invention, the solid dispersions according to the invention can be prepared by means of the solvent method. The active ingredient and the polymer are dissolved in organic solvents or solvent mixtures and the solution is then dried. The dissolution can also take place at elevated temperatures (30-150° C.) and under pressure.

Suitable organic solvents are dimethylformamide, tetrahydrofuran, methanol, ethanol, isopropanol, dimethylacetamide, acetone and/or dioxane or mixtures thereof. These solvents or solvent mixtures may additionally contain up to 20% by weight of water.

In principle, all types of drying are possible, such as, spray-drying, fluidized-bed drying, drum drying, freeze-drying, vacuum drying, belt drying, roller drying, carrier-gas drying, evaporation etc.

According to another embodiment of the invention, the solid dispersions are prepared by melt processes. The active ingredient is mixed with the polymer. By heating to temperatures of 50-180° C., the production of the solid dispersion takes place. Here, temperatures above the glass transition temperature of the polymer or the melting point of the active ingredient are advantageous. By adding a softening auxiliary, such as, for example, water, organic solvent, customary organic softeners, it is possible to correspondingly reduce the processing temperature. Of particular advantage are auxiliaries which can afterwards be very easily evaporated off again, i.e., having a boiling point below 180° C., preferably below 150° C.

According to a preferred embodiment, this type of preparation is carried out in a screw extruder. Which process parameters must be individually adjusted here can be determined by those skilled in the art by simple experiments in the scope of his or her conventional specialist knowledge.

According to a preferred embodiment, softeners are added during the melting. Preferred softeners are citric esters such as triethyl citrate or acetyl tributyl citrate, glycol derivatives such as polyethylene glycol, propylene glycol or poloxamers; castor oil and mineral oil derivatives; sebacate esters such as dibutyl sebacate), triacetin, fatty esters such as glycerol monostearate, fatty alcohols such as stearyl alcohol, fatty acids such as stearic acid, ethoxylated oils, ethoxylated fatty acids, ethoxylated fatty alcohols or vitamin E TPGS (tocopherol polyethylene glycol succinate). The softeners may be used in amounts of 0.1 to 40% by weight, preferably 1 to 20% by weight, based on the polymer.

The solid dispersions generated are amorphous. The amorphous state can be established by X-ray diffraction. The so-called “X-ray amorphous” state of the solid dispersions signifies that the crystalline proportion is less than 5% by weight.

The amorphous state can also by investigated with the aid of a DSC thermogram (Differential Scanning calorimetry). The solid dispersions according to the invention have no melting peaks but only a glass transition temperature, which depends also on the type of active ingredient used in the solid dispersions according to the invention. The glass transition temperatures are measured at a heating rate of 20 K/min.

In the course of preparation of the dosage forms according to the invention, customary pharmaceutical auxiliaries may optionally be processed at the same time. These are selected from the class of adsorbents, binders, disintegrants, dyes, fillers, flavorings or sweeteners, glidants, lubricants, preservatives, softeners, solubilizers, solvents or co-solvents, stabilizers (e.g., antioxidants), surfactants, or wetting agents.

The novel copolymers allow for inhibiting the recrystallization of active pharmaceutical ingredients in the aqueous media of the gastrointestinal tract after release of the active ingredient from the dosage form in which the active ingredient was present in the form of an amorphous solid dispersion of the active ingredient in the polymer matrix of the novel copolymers or in the form of a liquid solution of the active ingredient and the inventive copolymer in a suitable solvent vehicle system.

### EXAMPLES

The glass transition temperatures were calculated according to the Fox equation using the homopolymer T_{g} values given in Table 3.

$$\frac{1}{{T}_{G}} = {\sum }_{i}^{n} {x}_{i} \frac{1}{{T}_{G , i}}$$

xᵢ=mass fraction of the comonomer in the polymer

T_{G,i}=glass transition temperature of the homopolymer of the corresponding comonomer

T_{G}=glass transition temperature of the copolymer

TABLE 3

| | | Homopolymer | |
|---|---|---|---|
| Monomer | Abbreviation | T_{g} (° C.) | Reference |
| N-Vinylpyrrolidone | NVP | 175 | a |
| 2-Hydroxyethyl methacrylate | HEMA | 85 | a |
| tert-Butyl methacrylate | tBMA | 107 | a |
| Cyclohexyl methacrylate | CHMA | 104 | a |
| Isopropyl methacrylate | IPMA | 85 | a |
| N-Vinylcaprolactam | VCap | 145 | b |
| Acrylic acid | AA | 106 | a |
| Methacrylic acid | MAA | 228 | a |

a Glass transition temperatures of polymers, R. J. Andrews and E. A. Grulke, Polymer Handbook, (4^{th} Edition), 2003.
b F. Meeussen, Polymer 2000, 41, 8597-8602.

Residual Monomer Measurements:

The content of residual monomer, 2-pyrrolidone and azepan-2-one (ε-caprolactam) in synthesized polymer solutions were determined by reversed-phase liquid chromatography at 25° C. using UV detection at an absorbance wavelength of 205 nm. Chromatographic separation was achieved by using gradient elution. Quantification has been performed by external calibration. An aliquot of the sample was directly injected.

GPC-Method:

Polymer molecular weights were determined by size exclusion chromatography (SEC) at 35° C., using: dimethylacetamide containing 1 wt % trifluoroacetic acid and 0.5 wt % lithium bromide as eluent, narrow molecular weight distribution poly(methyl methacrylate) standards (commercially available from PSS Polymer Standard Solutions GmbH with molecular weights in the range from M=800 to M=2,200,000) and a differential refractive index (DRI) detector from DRI Wyatt Optilab DSP.

General Polymer Synthesis Procedure:

The used monomers can be divided into four different groups: i) the hydrophobic methacrylate group, consisting of tert-butyl methacrylate, isopropyl methacrylate and cyclohexyl methacrylate, ii) the N-vinyl lactam group, consisting of N-vinylpyrrolidone and N-vinylcaprolactam, iii) the acrylic carboxylic acid monomer group, consisting of acrylic acid and methacrylic acid and iv) 2-hydroxyethyl methacrylate.

A two-liter glass reactor, equipped with a mechanical stirrer, a condenser, a nitrogen sweep, a thermometer and inlets for the gradual additions of monomer and initiator, was charged with 300 grams isopropanol and 40% of the total N-vinyl lactam monomer amount. A monomer solution was prepared by dissolving one monomer from groups i and iii, 60% of the group ii monomer and, optionally, 2-hydroxyethyl methacrylate, in 240 grams of isopropanol. A total amount of 300 grams of monomer was used. An initiator solution was prepared by dissolving 4.0 grams tert-butyl peroxypivalate solution (75 wt % in mineral spirits) in 150 grams of isopropanol. A total of 10 wt % of the monomer solution was added to the reactor charge and the resulting solution was heated to a reactor temperature of 75° C. under stirring at 100 rpm. When the temperature reached 70° C., a total of 10 wt % of the initiator solution was added in 5 minutes. The remaining 90 wt % of the monomer and the initiator solutions were then added separately at constant feed rate to the stirring reactor charge over 4 and 6 hours respectively. During these additions, the temperature of the reaction mixture was maintained at 75° C. After the additions were complete, the reaction mixture was stirred at 75° C. for an additional hour and was then allowed to cool to ambient temperature. A sample was taken for residual monomer content measurements. Volatiles were removed and the product was dried overnight in a vacuum oven at 75° C. at 0.02 MPa. The used monomer amounts in grams are given in Table 4.

This general procedure was used for Polymers G1, G6-G10 and H1-3.

Polymers G2 and G5 were prepared analog to the described general polymer synthesis procedure with the exceptions that: the polymerization was performed at 80 instead of 75° C., 10 wt % of the initiator solution was added at 75 instead of 70° C. and 2,2′-azobis(2-methylbutyronitrile) (3.0 grams in the case of G2 and 3.5 grams in the case of G5) instead of tert-butyl peroxypivalate was used as polymerization initiator.

Polymers G3, I1-I3 were prepared analog to the described general polymer synthesis procedure with the exception that the entire amount of N-vinyl lactam monomer was included in the pre-feeding charge.

Polymers G4 was prepared analog to the described general polymer synthesis procedure with the exceptions that: a three-liter glass reactor was charged with 600 instead of 300 grams of isopropanol, a total amount of 600 instead of 300 grams of monomer was used, the monomer solution contained 510 instead of 240 grams of isopropanol, the initiator solution was prepared from 13.4 instead of 4.0 grams of tert-butyl peroxypivalate solution and 300 instead of 150 g of isopropanol, the initiator solution was added over 10 instead of 6 hours and the reaction mixture was kept at 75° C. for an additional 2 hours instead of 1 hour. After this, 250 grams of water were added, and the resulting solution was stirred for 2 hours.

N-Vinyl lactam monomers display lower reactivity than (meth)acrylic monomers. To improve their incorporation into the copolymer, at least a part of the N-vinyl lactam monomer was included in the pre-feeding charge.

The values in brackets in Table 4 give the monomer wt % values in the polymer. The amount of incorporated (meth)acrylic monomer was calculated by subtracting the used amount from the residual amount. The acidic conditions during the polymerization, resulting from the use of the carboxylic acid comonomer, increase the susceptibility of the N-vinyl lactam monomers to side reactions such as hydrolysis and addition of the solvent isopropanol to the alpha carbon atom of the olefinic double bond. The latter result in the formation of 1-(1-isopropoxyethyl)pyrrolidin-2-one and 1-(1-isopropoxyethyl)azepan-2-one in the cases of N-vinylpyrrolidone and N-vinylcaprolactam respectively. Unlike lactam monomer that is incorporated into the polymer, the N-vinyl lactam-isopropanol adducts and residual N-vinyl lactam monomer can be converted into the corresponding lactam in a post-polymerization hydrolysis. The amount of N-vinyl lactam monomer that was incorporated into the polymer chain was determined by the following procedure: i) water was added to a sample of the isopropanolic polymer solution (water/polymer solution in a 3:10 wt ratio), ii) this solution was stirred at 70° C. hours for 3 days, which leads to the complete hydrolysis of residual N-vinyl lactam monomer and N-vinyl lactam-isopropanol adducts, iii) the lactam content of these solutions was determined, iv) from this, the amount of N-vinyl lactam that was not incorporated in the polymer was calculated (NVP: (pyrrolidone (g)/85.1)×111.1, VCap: (caprolactam (g)/113.2)×139.2), iv subtracting this amount from the amount used in the polymerization affords the amount of N-vinyl lactam monomer incorporated in the polymer.

TABLE 4
Polymer composition.

| Polymer | tBMA | | CHMA | NVP | | VCap | | AA | | MAA | HEMA | |
|---|---|---|---|---|---|---|---|---|---|---|---|---|
| G1 | 135 | (46.1) | | 135 | (43.7) | | | 30 | (10.2) | | | |
| G2 | | | 120 (41.1) | 150 | (48.7) | | | 30 | (10.2) | | | |
| G3 | 90 | (33.8) | | | | 165 | (49.3) | 45 | (16.9) | | | |
| G4 | 180 | (30.4) | | 270 | (44.4) | | | 30 | (5.0) | | 120 | (20.2) |
| G5 | 45 | (16.1) | | | | 225 | (73.2) | 30 | (10.7) | | | |
| G6 | 75 | (25.4) | | 135 | (44.3) | | | 15 | (5.0) | | 75 | (25.3) |
| G7 | 99 | (33.4) | | 165 | (54.5) | | | | | 36 (12.1) | | |
| G8 | 45 | (16.0) | | | | 225 | (73.4) | 30 | (10.6) | | | |
| G9 | 90 | (30.5) | | 135 | (44.2) | | | 15 | (5.0) | | 60 | (20.3) |
| G10 | 105 | (35.7) | | 165 | (54.1) | | | 30 | (10.2) | | | |
| I1 | 198 | (68.7) | | 72 | (20.9) | | | 30 | (10.4) | | | |
| I2 | 165 | (61.9) | | | | 105 | (26.9) | 30 | (11.2) | | | |
| I3 | 186 | (67.5) | | | | 60 | (13.3) | 15 | (5.1) | | 39 | (14.1) |
| H1 | 30 | (10.3) | | 75 | (23.0) | | | 45 | (15.4) | | 150 | (51.3) |
| H2 | 21 | (7.1) | | 159 | (52.2) | | | 30 | (10.2) | | 90 | (30.5) |
| H3 | 15 | (5.1) | | 240 | (79.7) | | | 45 | (15.2) | | | |

Values refer to the amount of monomer in grams used in the synthesis procedure. The total amount of N-vinyl lactam monomer is given (sum of N-vinyl lactam in pre-feeding charge and in monomer feed). Values in brackets refer to the content of the monomer in the polymer in wt %.

Preparation of Amorphous Solid Dispersions Via Spray Drying

Celecoxib-Polymer Formulations (25 wt % Drug Loading):

Solid dispersions were composed of polymer and celecoxib. To prepare the formulations, 2.5 g of celecoxib and 7.5 g of polymer were dissolved in 190 g of methanol (5 wt % solids content). Spray drying was performed on a Büchi Mini Spray Dryer B-290 equipped with a 0.7 mm two-fluid nozzle under the following conditions:
Nitrogen flow rate . . . 35 m3/h
Inlet temperature . . . 85-105° C.
Outlet temperature . . . 50-70° C.
Atomizing pressure . . . 0.7 MPa
Liquid flow rate . . . 300 g/h

The product was collected using a cyclone. The drug content of the spray-dried formulations was determined by measuring the UV absorbance at 252 nm; the solid-state properties were analyzed using powder X-ray diffraction (PXRD):
Drug content (UV spectroscopy) . . . 24.6-27.5 wt %
Solid-state properties (PXRD) . . . X-ray amorphous

The same method was used to prepare amorphous solid dispersions of other APIs. Details are listed in Table 5.

TABLE 5
Preparation of amorphous solid dispersions.

| | Wavelength at | | |
|---|---|---|---|
| | which UV | | Solid state |
| | absorbance was | API loading | API properties |
| API | measured (nm) | (%) | determined by PXRD |
| Griseofulvin | 294 | 24.6-27.5 | X-ray amorphous |
| Danazol | 286 | 9.4-10.8 | X-ray amorphous |
| Naftopidil | 283 | 24.6-27.5 | X-ray amorphous |
| Telmisartan | 296 | 24.6-27.5 | X-ray amorphous |
| Probucol | 242 | 24.6-27.5 | X-ray amorphous |

In Vitro Release Experiments

Preparation of Fasted State Simulated Gastric Fluid (FaSSGF)

To prepare 1 L of FaSSGF solution, 2.00 g of sodium chloride was placed in a volumetric flask and dissolved in approximately 900 mL of water. The pH of the solution was adjusted to 1.6 by adding approximately 27 mL of a 1 M hydrochloric acid solution. Then, 0.06 g of FaSSIF/FeSSIF/FaSSGF powder (Biorelevant.com Ltd., London, United Kingdom) was added; the solution was diluted with water to 1 L.

Preparation of Concentrated Fasted State Simulated Intestinal Fluid (10×FaSSIF)

To prepare 1 L of 10×FaSSIF solution, 13.90 g of sodium hydroxide was placed in a volumetric flask and dissolved in approximately 900 mL of water. Then, 39.50 g of sodium dihydrogen phosphate, 43.90 g of sodium chloride, and 21.90 g of FaSSIF/FeSSIF/FaSSGF powder (Biorelevant.com Ltd., London, United Kingdom) were added. The solution was diluted with water to 1 L and allowed to stand for 2 h.

Dissolution Testing

In vitro dissolution tests were done to quantify the drug release and measure the maintenance of supersaturation. To this end, 262.5 mL of FaSSGF were filled into the dissolution vessels of an ERWEKA dissolution tester with mini glass vessels (stirring speed approximately 75 rpm). After a temperature of 37° C. had been reached, a defined amount of the spray-dried formulation (equivalent to a drug concentration of 0.14 mg/ml) was added. Samples of 3 mL were withdrawn after 5 min, 15 min and 30 min. After 30 min, 37.5 mL of 10×FaSSIF were added; the pH of the solution was adjusted to 6.8, if necessary. Additional samples of 3 mL were withdrawn after 5 min, 15 min, 30 min, 60 min, 90 min, 120 min, 180 min, 240 min, 300 min and 360 min. All samples were filtered through 0.45 μm PVDF syringe filters, and diluted with methanol or methanol/water (1:4 or 1:10, depending on the drug concentration). The concentration of the drug in solution was determined by UV spectroscopy using a calibration curve of the pure drug in methanol. To evaluate the performance of the polymer, the area under the concentration-time curve (AUC) was calculated as follows:

$$A U {C}_{0 \to {t}_{n}} = \frac{1}{2} {\sum }_{i = 1}^{n - 1} ( {C}_{{t}_{i}} + {C}_{{t}_{i + 1}} ) \cdot ( {t}_{i + 1} - {t}_{i} )$$

Cₜ=Concentration at time tin mg/mL, t=Time in min

TABLE 6
Polymer properties.

| Polymer | Mₙ^{a} | M_{w}^{a} | Tg (° C.)^{b} | SP (MPa^{1/2})^{c} |
|---|---|---|---|---|
| G1 | 8 450 | 29500 | 134 | 22.5 |
| G2 | 9780 | 30000 | 136 | 23.4 |
| G3 | 11500 | 32600 | 125 | 22.2 |
| G4 | 14500 | 44400 | 129 | 23.8 |
| G5 | 7140 | 21800 | 134 | 22.8 |
| G6 | 11200 | 45600 | 128 | 24.3 |
| G7 | 11300 | 55700 | 155 | 23.2 |
| G8 | 9760 | 24700 | 134 | 22.8 |
| G9 | 14800 | 52900 | 129 | 23.8 |
| G10 | 9100 | 29100 | 141 | 23.3 |
| I1 | 12500 | 37400 | 119 | 20.8 |
| I2 | 11000 | 33000 | 116 | 20.7 |
| I3 | 14100 | 43400 | 108 | 20.8 |
| H1 | 13200 | 42800 | 108 | 25.9 |
| H2 | 11100 | 37000 | 131 | 26.0 |
| H3 | 5910 | 17600 | 159 | 26.1 |
| A3^{d} | 11000 | 38900 | 119 | 21.9 |

^{a}Determined by GPC.
^{b}Calculated with Fox equation.
^{c}Calculated according to the Hoftyzer-Van Krevelen method.
^{d}From WO2019/121051

The results summarized in Table 7 show that of the tested copolymers, only those with a calculated solubility parameter (SP) value between 22.0 and 25.0 are effective as crystallization inhibitor for both celecoxib and danazol.

TABLE 7
Polymer performance for Celecoxib and Danazol.

| | Celecoxib release^{a} | Danazol release^{b} |
|---|---|---|
| Polymer | (%) | (%) |
| G1 | 92 | 97 |
| G2 | 97 | 86 |
| G3 | 87 | 95 |
| G4 | 97 | 87 |
| G5 | nd | 82 |
| G6 | nd | 78 |
| G7 | nd | 90 |
| I1 | nd | 2 |
| I2 | nd | 2 |
| I3 | nd | 0 |
| H1 | nd | 16 |
| H2 | nd | 16 |
| H3 | nd | 11 |
| A3^{c} | 44 | 25 |

^{a}Polymer/Celecoxib ratio = 3:1, Polymer/Danazol ratio = 9:1. Polymer spray formulation was incubated for 30 minutes in simulated gastric fluid before API dissolution was measured in simulated intestinal fluid (FaSSIF) over a 6 hour period. The given value is the percentage of theoretical maximum release which is complete API dissolution for the entire duration of the experiment.
^{c}From WO2019/121051, polymer partly neutralized with sodium hydroxide solution after polymerization.

Polymer spray formulation was incubated for 30 minutes in simulated gastric fluid before API dissolution was measured in simulated intestinal fluid (FaSSIF) over a 6 hour period. The given value is the percentage of theoretical maximum release which is complete API dissolution for the entire duration of the experiment. From WO2019/121051, polymer partly neutralized with sodium hydroxide solution after polymerization. Not determined=nd.

TABLE 8
Comparison of inventive polymers with HPMCAS-MF.^{a}

| | | | API release |
|---|---|---|---|
| | API release with | Inventive | with inventive |
| API | HPMCAS (%) | Polymer | polymer (%) |
| Griseofulvin | 51 | G8 | 100 |
| Naftopidil | 53 | G10 | 88 |
| Telmisartan | 84 | G8 | 98 |
| Danazol | 20 | G9 | 88 |
| Probucol | 42 | G10 | 94 |

^{a}Polymer/API ratio = 3:1. Polymer spray formulation was incubated for 30 minutes in simulated gastric fluid before API dissolution was measured in simulated intestinal fluid (FaSSIF) over a 6 hour period. The given value is the percentage of theoretical maximum release which is complete API dissolution for the entire duration of the experiment.

HPMCAS is available in different grades (L, M, H). Grades vary in the ratio between acetate and succinate groups (going from L to H, the number of acetate groups increases and the number of succinate groups decreases). [K. Ueda, K. Higashi, K. Yamamoto, K. Moribe, The effect of HPMCAS functional groups on drug crystallization from the supersaturated state and dissolution improvement, International Journal of Pharmaceutics 464 (2014) 205-213.] In the case of Griseofulvin and Probucol, drug release from L and H grade was investigated in addition to the release from the M Grade formulation given in Table 8. The inventive polymers were found to outperform all available HPMCAS grades (Griseofulvin: L 29% and H 53% release, Probucol L 59% and H 28% release).

1. A copolymer consisting of structural units derived from:
i) not less than 8% by weight on a total amount of all incorporated monomers of at least one hydrophobic methacrylate,
ii) at least one N-vinyl lactam monomer,
iii) 4 to 18% by weight on the total amount of all incorporated monomers of at least one acrylic carboxylic acid monomer, and
iv) optionally 2-hydroxyethyl methacrylate,
with the proviso that the total amount of incorporated monomers i) to iv) adds up to 100% by weight and the calculated solubility parameter SP of the copolymer is between 22.0 and 25.0 MPa^{1/2}.

2. The copolymer according to claim 1 consisting of structural units derived from:
i) not less than 8% by weight on the total amount of all incorporated monomers of at least one hydrophobic methacrylate selected from a group consisting of isopropyl methacrylate, tert-butyl methacrylate, and cyclohexyl methacrylate,
ii) at least one N-vinyl lactam monomer selected from the group consisting of N-vinylpyrrolidone and N-vinylcaprolactam,
iii) 4 to 18% by weight on the total amount of all incorporated monomers of at least one acrylic carboxylic acid monomer selected from the group consisting of acrylic acid and methacrylic acid, and
iv) optionally 2-hydroxyethyl methacrylate,
with the proviso that the total amount of incorporated monomers i) to iv) add up to 100% by weight
and a calculated solubility parameter SP of the copolymer is between 22.0 and 25.0 MPa^{1/2}.

3. The copolymer according to claim 1 consisting of structural units derived from:
i) 8 to 55% by weight on the total amount of all incorporated monomers of at least one hydrophobic methacrylate selected from a group consisting of isopropyl methacrylate, tert-butyl methacrylate, and cyclohexyl methacrylate,
ii) 10 to 88% by weight of N-vinyl lactam monomer, selected from the group consisting of N-vinylpyrrolidone and N-vinylcaprolactam
iii) 4 to 18% by weight on the total amount of all incorporated monomers of at least one acrylic carboxylic acid monomer selected from the group consisting of acrylic acid and methacrylic acid, and
iv) 0 to 40% by weight 2-hydroxyethyl methacrylate,
with the proviso that the total amount of incorporated monomers i) to iv) add up to 100% by weight and a calculated solubility parameter SP of the copolymer is between 22.0 and 25.0 MPa^{1/2}.

4. The copolymer according to claim 1, wherein the hydrophobic methacrylate is tert-butyl methacrylate.

5. The copolymer according to claim 1, wherein the hydrophobic methacrylate is cyclohexyl methacrylate.

6. The copolymer according to claim 1, wherein the N-vinyl lactam is N-vinyl pyrrolidone.

7. The copolymer according to claim 1, wherein the N-vinyl lactam is N-vinyl caprolactam.

8. The copolymer according to claim 1, having a calculated glass transition temperature in the range of 80 to 200° C.

9. The copolymer according to claim 1, having a calculated glass transition temperature in the range of 100 to 180° C.

10. The copolymer according to claim 1, having a weight average molecular weight in the range of 7,000 to 100,000 g/mol.

11. A process for manufacturing a copolymer according to claim 1 by radical polymerization of the monomers in the presence of a free radical initiator.

12. The process according to claim 11, wherein the polymerization is a solution polymerization.

13. The process according to claim 12, wherein the solution polymerization is carried out in an organic solvent.

14. The process according to claim 13 wherein the organic solvent is isopropanol.

15. A pharmaceutical dosage form, comprising a copolymer according to claim 1 and an active pharmaceutical ingredient with a solubility in water at standard conditions of less than 0.1% by weight, wherein the active ingredient is present in the amorphous form.

16. (canceled)

17. (canceled)

18. A method of inhibiting recrystallization of an active ingredient in a pharmaceutical dosage in an aqueous environment of a human or animal body comprising including a copolymer of claim 1 in the pharmaceutical dosage form as a recrystallization inhibitor, wherein the active ingredient has a solubility in water at standard conditions of less than 0.1% by weight, wherein the active ingredient is present in such dosage form in an amorphous form.

19. A method of inhibiting recrystallization of an agricultural active ingredient in an agricultural dosage form in soil comprising introducing a copolymer of claim 1 into the agricultural dosage form as a recrystallization inhibitor, wherein the agricultural active ingredient has a solubility in water at standard conditions of less than 0.1% by weight, wherein the agricultural active ingredient is present in such dosage form in an amorphous form.
